(19) United States
(12) Patent Application Publication
(10) Pub. No.: US 2023/0183758 A1
(43) Pub. Date: Jun. 15, 2023

(54) METHOD FOR PREPARING DIACYLGLYCEROL BY IMMOBILIZED SN-2 LIPASE

(71) Applicant: Shandong Academy of Agricultural Sciences, Jinan (CN)

(72) Inventors: TONGCHENG XU, Jinan (CN); JIAHUIZI KANG, Jinan (CN); WENJING WU, Jinan (CN); FANGLING DU, Jinan (CN); LINA LIU, Jinan (CN); AIZHEN ZONG, Jinan (CN); MIN JIA, Jinan (CN)

(21) Appl. No.: 18/077,328

(22) Filed: Dec. 8, 2022

(30) Foreign Application Priority Data
Dec. 14, 2021 (CN) 202111525352.5

(51) Int. Cl. C12P 7/20; C12N 9/18

(52) U.S. Cl. CPC C12P 7/20; C12N 9/18

(57) ABSTRACT

A method for preparing 1,3-diacylglycerol by immobilizing Sn-2 lipase. The present application obtains the Sn-2 lipase adsorbed by the macroporous resin by adsorbing the Sn-2 lipase on the resin, cross-links and immobilizes the Sn-2 lipase, and obtains 1,3-diglycerol by one-step hydrolysis with the lipase. The method improves the utilization times, hydrolysis activity and specificity of the Sn-2 lipase during hydrolysis, and determines the optimal parameters by improving the experimental conditions. This makes up for the shortcomings of immobilization and realizes the one-step hydrolysis of 1,3-diacylglycerol. Under optimal conditions, the hydrolase activity of immobilized lipase CAL-A is 1937.86U/g, and the immobilization rate can reach 94.49%, and has good operational stability and storage stability. Secondly, the DAG content can reach 36.12%, which is obviously superior to the current existing technology, and significantly reduces the production cost and market value of 1,3-DAG, providing favorable value for the industrial production of immobilized enzymes.

### CROSS-REFERENCE TO RELATED APPLICATIONS

This application claims the benefit and priority of Chinese patent application No. 202111525352.5, filed on Dec. 14, 2021, disclosure of which is hereby incorporated by reference in its entirety.

### TECHNICAL FIELD

The present application relates to the technical field of oil processing, in particular to a method for preparing 1,3-diacylglycerol by immobilizing Sn-2 lipase.

### BACKGROUND

Diacylglycerol (DG, DAG) is a structural lipid in oil. DAG is a trace component of natural vegetable oil and an endogenous intermediate product of fat metabolism in the body. It is a food ingredient generally recognized as safe (GRAS). In recent years, it has been found that dietary DAG has the functions of reducing visceral fat, inhibiting weight gain, and lowering blood lipids, so it has received extensive attention.

At present, for the preparation of diglycerides, most of the existing researches are about the lipases that hydrolyze and prepare 1,3- diacylglycerol, all of which are Sn-1,3-prone lipases or non-specific lipases, lack of study on Sn-2 prone lipases. The easiest way to prepare diacylglycerol is a two-step hydrolysis method. Although the existing technology can produce diglycerides by one-step hydrolysis, and the hydrolysis method is relatively simple, the degree of hydrolysis is difficult to control. If too much water is added, a large amount of oleic acid and MAG will be produced. If the hydrolysis is not enough, it cannot be converted into the required diacylglycerol.

### SUMMARY

In order to solve the above technical problems, the present application provides a method for immobilizing Sn-2 lipase, and using the immobilized Sn-2 lipase to hydrolyze 1,3-diglyceride in one step. This provides a new idea for the preparation of 1,3-diglyceride.

The method of the present application improves the utilization times, hydrolysis activity and specificity of the Sn-2 lipase during hydrolysis, and determines the optimal parameters by improving the experimental conditions. This makes up for the shortcomings of immobilization and realizes the one-step hydrolysis of 1,3-diacylglycerol.

The present application is achieved through the following technical solutions:

S1 Adsorb Sn-2 lipase with resin: pipette original enzyme solution of Sn-2 lipase in a centrifuge tube, add pretreated wet carrier macroporous resin, shake and absorb in a water bath, and a reaction is completed after dispersion and uniformity, air-dry overnight after suction filtration, and dry to obtain Sn-2 lipase adsorbed by macroporous resin;

S2 Cross-linking and immobilization of Sn-2 lipase: after resin adsorption is completed, add a cross-linking agent, oscillate for cross-linking, filter, wash with buffer, filter, dry, and refrigerate to obtain immobilized Sn-2 lipase macroporous resin;

S3 Hydrolysis: Add immobilized Sn-2 lipase macroporous resin to the oil raw material, and obtain 1,3-diglycerol by one-step hydrolysis.

Preferably, the Sn-2 lipase is Candida antarctica lipase A.

Preferably, in S1, a pretreatment method of the macroporous resin is: soak in 95% absolute ethanol for 24h, stir continuously to make it dissolve completely, vacuum filtration, rinse with distilled water until the water is no longer turbid and there is no obvious ethanol smell, wash away the residual impurities and absolute ethanol, soak in deionized water and seal at 4° C. for storage for later use.

Preferably, in S1, the model of macroporous resin is any one of RDK09, DA201, LXT-008, HPD700, HPD100, X-5, HPD750.

Preferably, in S1, conditions for water-bath shaking adsorption are: shake at 250r/min on a water-bath shaker for 12h at 25-70° C.

Preferably, in S1, conditions of the adsorption are: pH of the buffer solution is 6.0˜10, an adsorption temperature is 50.5° C., the amount of enzyme is 200 mg/g, and an adsorption time is 10˜11h.

Preferably, in S2, the cross-linking agent is one of0.5%, 1.0% glutaraldehyde, glyoxal, oxalic acid diglycidyl ether, 1,4 butyl glycol diglycidyl ethers, wherein the volume concentration of the cross-linking agent is 0.5-1.0%.

Preferably, in S2, conditions of the cross-linking are: cross-linking temperature 49-50° C., cross-linking time 1˜2h, cross-linking agent dosage 0.4˜0.8% (volume).

Preferably, the buffer is one of disodium hydrogen phosphate-citric acid buffer, potassium dihydrogen phosphate-sodium hydroxide buffer, disodium hydrogen phosphate-sodium dihydrogen phosphate buffer, barbiturate-hydrochloric acid buffer, tris hydrochloride buffer, glycine-sodium hydroxide buffer.

Preferably, in S3, the oil raw material is animal oil and vegetable oil.

Preferably, the oil raw material is high oleic sunflower oil.

Preferably, in S3, conditions of the hydrolysis are: hydrolysis temperature 60˜65° C., hydrolysis time 7˜8h, the amount of enzyme 7˜9% (volume), the amount of water added 3˜4% (volume).

Further, adsorption and immobilization of free lipase CAL-A: firstly, the medium-polarity macroporous resin HPD750 with the highest relative enzyme activity is screened out from 7 kinds of macroporous resins, and then the free CAL-A lipase is immobilized by adsorption method using it as a carrier, the buffer solution with the highest immobilization relative enzyme activity and immobilization rate is found to be disodium hydrogen phosphate-citric acid. Finally, in order to obtain the best adsorption conditions, this application carried out single factor and response surface experiments on the four factors of buffer pH, adsorption temperature, enzyme amount, and adsorption time. At the same time, combined with factors such as cost, the optimal adsorption conditions are finally determined to be buffer pH=8.00, adsorption temperature 50.5° C., enzyme dosage 200 mg/g, and adsorption time 10.1 h, so that the immobilized enzyme CAL-A could be obtained, and its enzyme activity is 1610.67 U/g, the immobilization rate reached 82.39%.

Further, the immobilized lipase is immobilized by the CAL-A adsorption-cross-linking method: Five different cross-linking reagents were selected to compare the enzyme activities of the immobilized enzymes. Glyoxal is selected as the best cross-linking agent. Single factor and response surface experiments were carried out on the three factors of cross-linking time, cross-linking temperature and cross-linking agent dosage. Finally, the optimal cross-linking conditions were determined as follows: under the conditions of cross-linking temperature 49.4° C., cross-linking time 1.76h, cross-linking agent dosage 0.6%, the immobilized lipase CAL-A is obtained, and its hydrolase activity is 1937.86U/g, the immobilization rate reached 94.49%.

Further, the results of research on the properties of the free enzyme and the immobilized enzyme CAL-A enzyme: In terms of temperature: the optimum temperature of the free enzyme CAL-A is 70° C., and the optimum temperature of the macroporous resin adsorption-cross-linked immobilized enzyme is 60° C. Although the optimum temperature is lower than that of the free enzyme CAL-A, the thermal stability is improved. The macroporous resin adsorption-cross-linked immobilized enzyme can still maintain 90% of the activity of the immobilized CAL-A at 80° C. The free enzyme is only 64.71%. In terms of pH value: in an environment with a pH value of 4 to 12, it is found that the most suitable pH value of the free enzyme is 8, while the most suitable pH value of the immobilized enzyme is 8.5, and the acid-base stability is also improved. In terms of organic solvents: ethanol, petroleum ether, n-hexane, and acetic acid have obvious inhibitory effects on hydrolysis activity, while ethyl acetate, n-butanol, and tert-butanol have activation effects on immobilized enzymes. In terms of metal ions and surfactants: metal ions Fe²⁺ can activate the hydrolysis of immobilized enzymes. During the experiment, contact with Zn²⁺, Mn²⁺, Ba²⁺ should be avoided. 0.0005% EDTA in the surfactant can activate lipase, while 0.001% EDTA can inhibit lipase, and the immobilized enzyme has good operation stability and storage stability.

Further, the immobilized CAL-A is hydrolyzed to obtain 1,3- diacylglycerol: Using high oleic acid sunflower oil as raw material, 1,3- diacylglycerol is prepared by hydrolysis with macroporous resin adsorption-cross-linking method. The present application uses the HPLC-ELSD method to analyze the content of diacylglycerol in oils. At the same time, single factor and response surface experiments were carried out on the four factors of hydrolysis time, hydrolysis temperature, the amount of enzyme and the amount of water, and finally the optimal hydrolysis conditions were determined as hydrolysis temperature is 62.4° C., hydrolysis time is 7.5h, and the amount of enzyme is 8.1%, the amount of water is 3.72%, and the DAG content can reach 36.12% under this condition.

The beneficial effect of the present application is:

The present application innovatively prepares 1,3- diacylglycerol by immobilizing Sn-2 lipase, which provides a new idea for the preparation of 1,3- diacylglycerol. In the present application, the Sn-2 lipase is first immobilized, and then it could be used as a hydrolase to hydrolyze oil in one step to prepare 1,3- diacylglycerol. This is because immobilization can improve the utilization times, hydrolysis activity and specificity of Sn-2 lipase during hydrolysis. One-step hydrolysis can significantly reduce the production cost and market value of 1,3-DAG, and provide favorable value for the industrial production of immobilized enzymes.

Further, the present application has outstanding technical advantages. The optimal process parameters were found by single factor and response surface experiments. Under optimal conditions, the hydrolase activity of the immobilized lipase CAL-A is 1937.86U/g, and it has good operation stability and storage stability. The DAG content can reach 36.12%, which is obviously better than the prior art.

### BRIEF DESCRIPTION OF THE DRAWINGS

FIG. 1-A is the relative enzyme activity and immobilization rate of immobilized lipase prepared by different buffers;

FIG. 1-B is the relative enzyme activity and immobilization rate of immobilized lipase prepared at different buffer pHs;

FIG. 1-C is the relative enzyme activity and immobilization rate of immobilized lipase prepared at different adsorption temperatures;

FIG. 1-D is the relative enzyme activity and immobilization rate of immobilized lipase prepared at different enzyme amounts;

FIG. 1-E is the influence of different enzyme amounts on the recovery rate of immobilized lipase enzyme activity;

FIG. 1-F is the influence of different adsorption times of macroporous resins on the relative enzyme activity and immobilization rate of immobilized lipase;

FIG. 2 is the relative enzyme activity, immobilization rate and enzyme activity recovery rate of different wet carrier macroporous resins;

FIG. 3-A is the influence of different cross-linking agents on the relative enzymatic activity of immobilized lipase;

FIG. 3-B is the influence of different temperatures on the relative enzyme activity and immobilization rate of immobilized lipase;

FIG. 3-C is the influence of different cross-linking times on the relative enzyme activity and immobilization rate of immobilized lipase;

FIG. 3-D is the influence of different glyoxal concentrations on the relative enzyme activity and immobilization rate of immobilized lipase;

FIG. 4-A is the relative enzyme activity of free enzyme and immobilized enzyme under optimum temperature;

FIG. 4-B is the relative enzyme activity of free enzyme and immobilized enzyme after 2 hours;

FIG. 4-C is the relative enzyme activity of free enzyme and immobilized enzyme under optimum pH;

FIG. 4-D is the influence of the relative enzyme activity of immobilized enzyme and free enzyme after 2 hours;

FIG. 4-E is the influence of different organic solvents (10%) on relative enzyme activity;

FIG. 4-F is the influence of different organic solvents (20%) on the relative enzyme activity;

FIG. 4-G is the influence of different metal ions (1 mmol/L) on the relative enzyme activity;

FIG. 4-H is the influence of different metal ions (5 mmol/L) on the relative enzyme activity;

FIG. 4-I is the influence of different metal ions (10 mmol/L) on relative enzyme activity;

FIG. 4-J shows the influence of different surfactants (0.0001%) on relative enzyme activity;

FIG. 4-K is the influence of different surfactants (0.0005%) on the relative enzyme activity;

FIG. 4-L is the influence of different surfactants (0.001%) on relative enzyme activity;

FIG. 4-M is the broken line graph of the change in enzyme activity of immobilized enzyme and free enzyme sealed preservation 60d;

FIG. 4-N is the broken line graph of the change in relative enzyme activity of immobilized enzymes after repeated washing with buffer,

FIG. 5-A is the influence of different hydrolysis time on DAG content;

FIG. 5-B is the influence of different hydrolysis temperatures on the DAG content;

FIG. 5-C is the influence of different enzyme amounts on the DAG content;

FIG. 5-D is the influence of different water amounts on the DAG content.

### DETAILED DESCRIPTION

The present application is described below through specific embodiments. Unless otherwise specified, the technical means used in the present application are methods known to those skilled in the art. In addition, the embodiments should be understood as illustrative rather than limiting the scope of the application, the spirit and scope of which is defined only by the claims. For those skilled in the art, on the premise of not departing from the spirit and scope of the present application, various changes or modifications to the material components and dosage in these embodiments also belong to the protection scope of the present application.

The reagents, instruments and manufacturers used in the present application are shown inTable 1 and Table 2:

TABLE 1
Specific information of main reagents and drugs in the experiment

| Name or model | Manufacturer |
|---|---|
| HPD-750 | Zhengzhou Hecheng New Material |
| | Technology Co., Ltd. |
| Absolute ethanol | Sinopharm Chemical Reagent Co., Ltd. |
| Free lipase CALA | Novozymes (China) Biotechnology Co., Ltd. |
| potassium chloride | Sinopharm Chemical Reagent Co., Ltd. |
| Sodium chloride | Sinopharm Chemical Reagent Co., Ltd. |
| magnesium chloride | Sinopharm Chemical Reagent Co., Ltd. |
| ferrous sulfate | Sinopharm Chemical Reagent Co., Ltd. |
| ferric chloride | Sinopharm Chemical Reagent Co., Ltd. |
| calcium chloride | Sinopharm Chemical Reagent Co., Ltd. |
| Manganese sulfate | Sinopharm Chemical Reagent Co., Ltd. |
| Zinc chloride | Sinopharm Chemical Reagent Co., Ltd. |
| barium chloride | Sinopharm Chemical Reagent Co., Ltd. |
| Sodium dihydrogen phosphate | Sinopharm Chemical Reagent Co., Ltd. |
| citric acid | Sinopharm Chemical Reagent Co., Ltd. |
| Methanol | Tianjin Fuyu Fine Chemical Co., Ltd. |
| ethanol | Tianjin Fuyu Fine Chemical Co., Ltd. |
| n-Hexane | Shandong Yuwanghetianxia New Material |
| | Co., Ltd. |
| Acetonitrile | Shandong Yuwanghetianxia New Material |
| | Co., Ltd. |
| acetone | Lihuayi weiyuan Chemical Co., Ltd. |
| Butanol | Tianjin Fuyu Fine Chemical Co., Ltd. |
| Isopropanol | Sinopharm Chemical Reagent Co., Ltd. |
| Isooctane | Sinopharm Chemical Reagent Co., Ltd. |
| n-heptane | Sinopharm Chemical Reagent Co., Ltd. |
| petroleum ether | Sinopharm Chemical Reagent Co., Ltd. |
| tert-butanol | Sinopharm Chemical Reagent Co., Ltd. |
| Glacial acetic acid | Sinopharm Chemical Reagent Co., Ltd. |
| ethyl acetate | Shandong Yuwanghetianxia New Material |
| | Co., Ltd. |
| TritonX-100 | Beijing Coollab Technology Co., Ltd. |
| SDS | Beijing Solarbio Science&Technology |
| | Co., Ltd. |
| Tween 80 | Biofrox company (German) |
| Tween 20 | Shang hai Macklin Biochemical Co., Ltd. |
| EDTA | Beijing Solarbio Science&Technology |
| | Co., Ltd. |
| Tris(hydroxymethyl)aminomethane | Beijing Solarbio Science&Technology |
| Tirs | Co., Ltd. |
| p-Nitrophenol (P-NP) | Shang hai Macklin Biochemical Co., Ltd. |
| p-Nitrophenol Palmitate (P-NPP) | Shanghai Yuanye Biotechnology Co., Ltd. |
| Coomassie Brilliant Blue (G-250) | Beijing Coollab Technology Co., Ltd. |
| Disodium hydrogen phosphate | Sinopharm Chemical Reagent Co., Ltd. |
| (AR) | |
| Citric acid | Sinopharm Chemical Reagent Co., Ltd. |
| Macroporous resin HPD750 | Zhengzhou Hecheng New Material |
| | Technology Co., Ltd. |
| Nissin High Oleic Sunflower Oil | Jinli Oil (Suzhou) Co., Ltd, |
| Fatty acid methyl ester mixed | Sigma-Aldrich (Shanghai) Trading Co., Ltd. |
| standard | |
| Boron trichloride methanol | Shanghai Yuanye Biotechnology Co., Ltd. |
| solution | |
| n-Hexane | Shandong Yuwanghetianxia New Material |
| | Co., Ltd. |
| Isopropanol | Shandong Yuwanghetianxia New Material |
| | Co., Ltd. |
| ethyl acetate | Shandong Yuwanghetianxia New Material |
| | Co., Ltd. |
| Acetonitrile | Shandong Yuwanghetianxia New Material |
| | Co., Ltd. |

Note:
Unless otherwise specified, all reagents are analytically pure.

TABLE 2
Main instruments in the experiment

| Experimental Apparatus | Manufacturer and model |
|---|---|
| BCD-215KALM Refrigerator | Qingdao Haier Co., Ltd. |
| Electric blast drying oven | Shanghai Xinmiao Medical Device |
| | Manufacturing Co., Ltd. |
| electronic analytical balance | Swiss Mettler-Toledo Group |
| Instrument constant | Shanghai Shuia Instrument Co., Ltd. |
| temperature water bath | |
| BIOTEK microplate reader | Highland Park, Winorski, Vermont, USA |
| Electronic analytical balance | Swiss Mettler-Toledo Group |
| Instrument constant | Shanghai Shula Instrument Co., Ltd. |
| temperature water bath | |
| SHA-A constant temperature | Tianjin Sateli Experimental Analysis |
| water bath oscillator | Instrument Manufacturing Factory |
| AS20500A Ultrasonic | Tianjin Autosines Instrument Co., Ltd. |
| Cleaning Machine | |
| DHG-92435-III Electric | Shanghai Xinmiao Medical Device |
| Heating Constant | Manufacturing Co., Ltd. |
| Temperature Drying Oven | |
| BCD-215KALM Refrigerator | Qingdao Haier Co., Ltd. |
| pH meter | Mettler-Toledo Instruments (Shanghai) |
| | Co., Ltd. |
| SHK-IIIS circulating water | Zhengzhou Ketai Experimental Equipment |
| type multi-purpose vacuum | Co., Ltd. |
| pump | |
| Agilent 7890N Gas | American Agilent Technologies Co.. Ltd. |
| Chromatograph | |

### Embodiment 1

Preparation of diacylglycerol by immobilized Sn-2 lipase, the steps are as follows:

1) Soak different types of macroporous resins in 95% absolute ethanol for 24 hours, stir continuously to dissolve them completely, vacuum filter, rinse with distilled water until the water is no longer turbid and has no obvious ethanol smell, wash away the residual impurities and absolute ethanol, soak the processed macroporous resin in deionized water, seal it and store it at 4° C. for later use;

2) Weigh 1 g of free Candida antarctica lipase, use 12 mL of 50 mmol/L, pH=8.0 phosphate buffer, centrifuge at 10000 r/min for 10 min to facilitate the removal of impurities, and take the supernatant solution and store it in a refrigerator at 4° C. for later use;

3) Pipette 10 mL of the original enzyme solution into a 50 mL centrifuge tube, add 1 g of pretreated HPD750 wet carrier macroporous resin, shake and adsorb on a water bath shaker at 50.5° C. and 250r/min for 10.1h, and the reaction is completed after the dispersion is uniform. After suction filtration, dry overnight, dry at 40° C. for 4 hours, and refrigerate at 4° C. for later use;

4) Immediately after the immobilization of lipase by macroporous resin adsorption, add 0.6% concentration (v/v) of cross-linking agent, the cross-linking temperature is 49.4° C., the oscillator is oscillating at 200 rpm for 1.76h, and the reaction is completed, filter and wash with a buffer solution with a pH of 8.0, filter to obtain the immobilized enzyme, dry overnight at 40° C. for 4 hours, and place in a refrigerator at 4° C. for later use;

5) Add immobilized Sn-2 lipase macroporous resin with a volume concentration of 8.1% to high oleic sunflower oil, add 3.72% water, hydrolyze at 62.4° C. for 7.5 hours, and obtain 1,3- diacylglycerol in one step.

### Embodiment 2

In order to determine the influence of the screening of macroporous resin on the preparation method of the present application and find the optimal conditions, the application has carried out single factor and response surface experiments, and based on the principle of single variable, relevant tests have been done, as follows:

1) Screening of macroporous resin

Weigh 1 g of free Candida antarctica lipase A, use 12 mL of pH=7 phosphate buffer, pipette 10 mL of the original enzyme solution, respectively add 1 g of wet carrier macroporous resin pretreated by RDK09, DA201, LXT-008, HPD700, HPD100, X-5, HPD750, shake at 30° C., 200 r/min water bath shaker for 12 h, disperse evenly and then suction filter, dry overnight, dry at 40° C. for 4 hours, and refrigerate at 4° C. for later use.

Optimize the macroporous resin after conducting pro-experiments with reference to the literature. First, soak different types of macroporous resins in 95% absolute ethanol for 24 hours, stir continuously to dissolve them completely, vacuum filter, rinse with distilled water until the water is no longer turbid and has no obvious ethanol smell, and wash away the remaining impurities and absolute ethanol, soak the processed macroporous resin in deionized water, seal it and store it at 4° C. for future use.

Measuring the immobilization rate and enzyme activity recovery rate of different macroporous resin carriers, the steps are as follows:

S1: The ability of the carrier to adsorb protein molecules is expressed by the immobilization rate

$$Immobilization rate = \frac{A 1 - A 2}{A 1} \times 100 \%$$

A₁: protein content in the solution before fixation/ug

A₂: Protein Content in Solution after Fixation/Ug

The protein concentration of immobilized lipase is determined by Coomassie Brilliant Blue method by literature method, the steps are as follows:

Dissolve 100 mg of Coomassie Brilliant Blue G-250 in 50 mL of 95% ethanol solution, add 100 mL of85% phosphoric acid solution, and dilute to 1000 mL with deionized water. Accurately weigh 0.001 g bovine serum albumin as standard protein, add 10 mL deionized water to prepare protein solution. Add the standard bovine serum albumin and Coomassie brilliant blue solution in the table into the test tube in sequence, shake well, and measure the absorbance at ODm with a microplate reader after standing for 5 minutes. Draw a standard curve with the absorbance as the ordinate and the standard bovine serum albumin content as the abscissa.

Take 1 mL of the sample solution to be tested, add 5 mL of the prepared Coomassie Brilliant Blue reagent, shake well, let it stand for 5 min, and measure its absorbance at OD₅₉₅ using a microplate reader.

TABLE 3
Drawing of standard curve for bovine serum albumin

| | Bovine serum albumin concentration (ug/mL) | | | | | | | | | | |
|---|---|---|---|---|---|---|---|---|---|---|---|
| | 0 | 1 | 2 | 3 | 4 | 5 | 6 | 7 | 8 | 9 | 10 |
| Bovine serum | 0 | 0.1 | 0.2 | 0.3 | 0.4 | 0.5 | 0.6 | 0.7 | 0.8 | 0.9 | 1.0 |
| albumin | | | | | | | | | | | |
| standard | | | | | | | | | | | |
| solution (mL) | | | | | | | | | | | |
| Coomassie | 5.0 | 5.0 | 5.0 | 5.0 | 5.0 | 5.0 | 5.0 | 5.0 | 5.0 | 5.0 | 5.0 |
| brilliant blue | | | | | | | | | | | |
| solution (mL) | | | | | | | | | | | |

S2: Recovery Rate of Enzyme Activity

$$Recovery rate of enzyme activity = \frac{Total activity of immobilized enzyme}{Total activity of free enzymes used for immobilization \times immobilization rate} \times 100 \%$$

Note: The definition of relative enzyme activity: in the same group of experimental values, the highest point of activity is recorded as 100%, and the ratio of other experimental points to this point is the relative enzyme activity, which is generally expressed as a percentage.

Correspondingly, the enzyme activity determination method of the present application, the steps are as follows:

Use P-NPP to measure the hydrolysis activity of lipase. During the hydrolysis process, the generated p-nitrophenol has the largest characteristic absorption peak near 410 nm and appears yellow in water, so the lipase activity can be characterized by the amount of generated p-nitrophenol, and the sensitivity is high. The determination method is as follows:

Weigh 0.1 g of lipase, dilute to 100 mL with pH-8 phosphate buffer, and refrigerate for later use. Prepare a standard solution of LP-NPP with a concentration of 0.1 mg/mL, weigh 0.0520 g p-np, dilute to 500 mL with tris-HC1 buffer with pH=7, respectively take 0, 0.1, 0.2, 0.3, 0.4, 0.5, 0.6, 0.7, 0.8, 0.9, 1.0 mL of P-NP standard solution in the test tube, respectively dilute to 10 mL by buffer, measure the absorbance value OD404, and OD404 is the abscissa, and the amount of P-NPP substance is the ordinate to draw a standard curve.

Prepare 1 mmol/L P-NPP substrate solution: Weigh 0.0378 g of P-NPP, add 1 mL trionX-100 and 50 mmol/L Tris-HCL buffer to dissolve, then add SmL isopropanol, and then use 50 mmol/L TIRS-HCL to dilute it to 100 mL and store it at 4° C. for later use.

Take 0.5 mL of 1 mmol/L P-NPP solution, 0.5 mL of 50 mmol/L Tris-HCL buffer, and 1 mL of the total reaction system, mix well and keep warm at 37° C. for 5-10 min, add 50 ul of the enzyme solution to be tested that is suitable for dilution, take it out quickly after 10 minutes of reaction, dilute to 3.75 mL, and place it at −20° C. for 5 min to terminate the reaction, measure the absorbance value at 404 nm, use the enzyme activity under the same conditions as a blank, and calculate the hydrolysis activity from the P-NPP standard curve.

TABLE 4
P-NPP standard curve drawing

| | P-NPP concentration (ug/mL) | | | | | | | | | | |
|---|---|---|---|---|---|---|---|---|---|---|---|
| | 0 | 1 | 2 | 3 | 4 | 5 | 6 | 7 | 8 | 9 | 10 |
| P-NPP | 0 | 0.1 | 0.2 | 0.3 | 0.4 | 0.5 | 0.6 | 0.7 | 0.8 | 0.9 | 1.0 |
| standard | | | | | | | | | | | |
| solution (mL) | | | | | | | | | | | |
| Buffer | 10 | 9.9 | 9.8 | 9.7 | 9.6 | 9.5 | 9.4 | 9.3 | 9.2 | 9.1 | 9.0 |

Under the above conditions, the amount of enzyme needed to hydrolyze P-NPP to produce 1 mmol p-nitrophenol per minute is defined as one enzyme activity unit (U).

$$U = \frac{( α \times Δ OD + b ) \times F}{10 \times T \times 0.1} \qquad ( Formula 1 )$$

(α×ΔOD+b): The amount of p-nitrophenol in the corresponding P-NP standard curve under the absorbance

F: dilution ratio of lipase solution

10: Conversion coefficient for 1 mL reaction system

T: reaction time

0.1: volume of enzyme solution

S3: Data processing

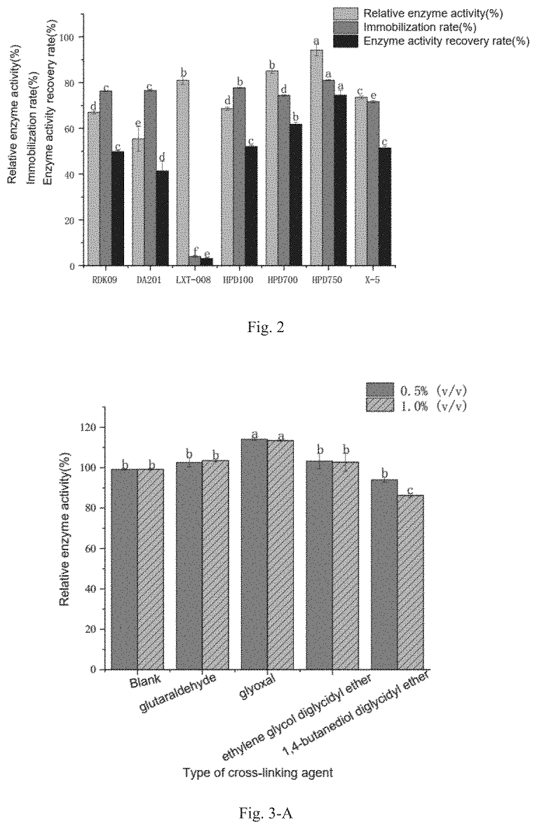

As shown in FIG. 2, the relative enzyme activity, immobilization rate, and enzyme activity recovery rate of HPD750 are higher than those of other macroporous resins, so the optimal macroporous wet carrier for the present application is HPD750.

### Embodiment 3

In order to determine the influence of buffer pH, adsorption temperature, enzyme amount, and adsorption time on the preparation method of the present application, the present application carried out single factor and response surface experiments on these four factors, and combined with factors such as cost to find the optimal conditions. The relevant experiments are as follows:

1) Influence of Buffer Type on Immobilization

Weigh 1 g of free Candida antarctica lipase A, add 12 mL of disodium hydrogen phosphate-citric acid buffer, potassium dihydrogen phosphate-sodium hydroxide buffer, disodium hydrogen phosphate-sodium dihydrogen phosphate buffer, barbiturate-hydrochloric acid buffer, Tris-HC1 buffer, glycine-sodium hydroxide buffer, pipette 10 mL of the original enzyme solution, add 1 g of pretreated wet carrier macroporous resin, shake in a water-bath shaker at 30° C. and 250r/min for 12h, disperse evenly, filter with suction, dry overnight, dry at 40° C. for 4h, and refrigerate at 4° C. for later use.

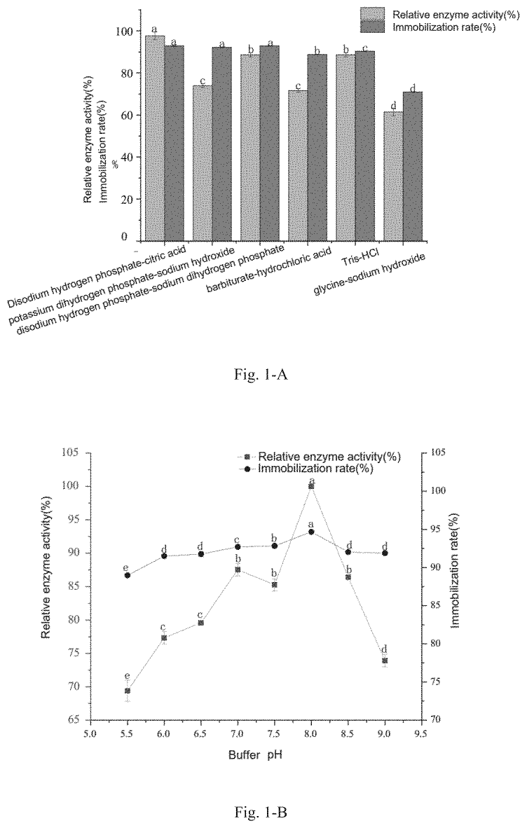

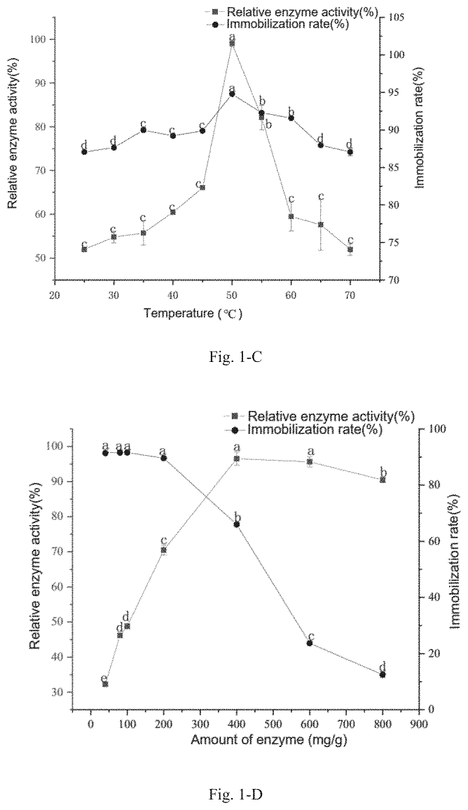

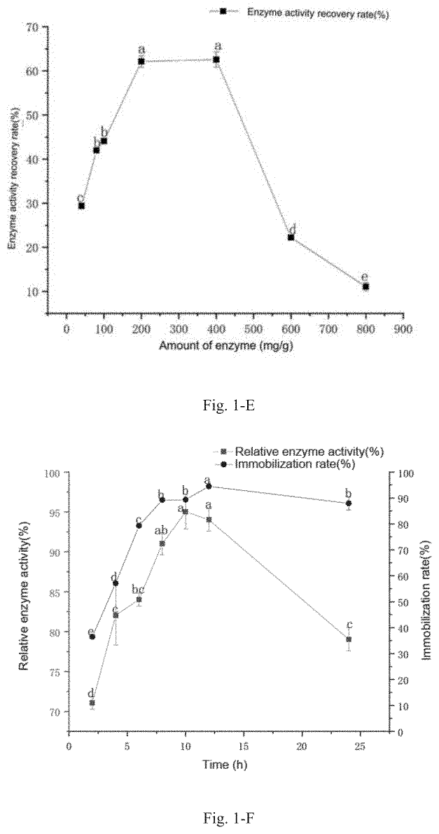

Determine the relative enzyme activity and immobilization rate of immobilized lipase prepared by different buffers (the test method is the same as that in Embodiment 2), data processing, as shown in FIG. 1-A, the relative enzyme activity and immobilization rate of disodium hydrogen phosphate-citric acid are higher than other buffers, both above 90%, which is the optimal solution.

2) Influence of Buffer pH on Immobilization

Weigh 1 g of free Candida antarctica lipase A, prepare 12 mL of 50 mmol/L disodium hydrogen phosphate-citric acid buffer with pH values of5.5, 6.0, 6.5, 7.0, 7.5, 8.0, 8.5, and 9.0, pipette 10 mL of the original enzyme solution, add 1 g of pretreated wet carrier macroporous resin, shake in a water bath shaker at 30° C. and 250r/min for 12h, disperse evenly, filter with suction, dry overnight, and dry at 40° C. for 4h, refrigerate at 4° C. for later use.

Determine the relative enzyme activity and immobilization rate of immobilized lipase prepared at different buffer pHs (the test method is the same as that in Embodiment 2), data processing, as shown in FIG. 1-B, the relative enzyme activity and immobilization rate at pH-8 are all higher than other pHs, all above 90%, which is the optimal solution.

3) Influence of Adsorption Temperature on Immobilization

Weigh 1 g of free Candida antarctica lipase A, prepare 12 mL of disodium hydrogen phosphate-citric acid buffer with a concentration of 50 mmol/L and a pH value of 8.0, pipette 10 mL of the original enzyme solution, and add 1 g of pretreated wet carrier Macroporous resin, shake in a water bath shaker at 25, 30, 35, 40, 45, 50, 55, 60, 65, 70° C., 250r/min for 12h, disperse evenly, filter with suction, dry overnight, dry at 40° C. for 4 h, and refrigerate at 4° C. for later use.

Determine the relative enzyme activity and immobilization rate of immobilized lipase prepared at different adsorption temperatures (the test method is the same as that in Embodiment 2), data processing, as shown in FIG. 1-C, the relative enzyme activity and immobilization rate at 50° C. are both high than other adsorption temperatures, all above 90%, which is the optimal solution.

4) Influence of Enzyme Amount on Immobilization

Prepare 12 mL of disodium hydrogen phosphate-citric acid buffer with a concentration of 0.05 mol/L and a pH value of 8.0, and add free Candida antarctica lipase A to a solution with enzyme activity of 40, 80,100, 200, 400, 600, 800 (ug/g), pipette 10 mL of the original enzyme solution, add 1 g of pretreated wet carrier macroporous resin, shake in a water bath shaker at 50° C. and 250r/min for 12h, disperse evenly, filter with suction, dry overnight, and dry at 40° C. for 4h, refrigerate at 4° C. for later use.

Determine the relative enzyme activity, recovery rate of enzyme activity and immobilization rate of immobilized lipase prepared with different enzyme amounts (the test method is the same as that in Embodiment 2), data processing, as shown in FIG. 1-D and FIG. 1-E, when the enzyme amount is 400 mg/g, it is the maximum relative enzyme activity, recovery rate of enzyme activity and immobilization rate, combined with cost factors, 200 mg/g is the best choice.

5) Influence of Adsorption Time on Immobilization

Add a solution of free Candida antarctica lipase A with an enzyme activity of200 ug/g, prepare 12 mL of disodium hydrogen phosphate-citric acid buffer with a concentration of0.05 mol/L and a pH value of8.0, centrifuge, and pipette 10 mL of the original enzyme solution, add 1 of pretreated wet carrier macroporous resin, shake in a water bath shaker at 50° C. and 250r/min for 10 h, 11h, 12h, 13h, 14h, 24h, then disperse evenly, then suction with filter, and dry overnight, and dry at 40° C. for 4 h, and refrigerate at 4° C. for later use.

Determine the relative enzyme activity and immobilization rate of immobilized lipase prepared at different adsorption times (the test method is the same as that in Embodiment 2), data processing, as shown in FIG. 1-F, combined with production cost factors, it is the best choice when adsorbed for 10 h.

6) Optimization of Immobilization Conditions by Response Surface Experiments

According to the results of the single factor experiment, the Box-Behnken design principle is used to conduct a three-factor three-level response surface analysis test on the buffer pH, buffer temperature, and adsorption time to further explore the influence of the three factors on the activity of immobilized lipase, and finally determine the best immobilization conditions, and do three parallel experiments for each group.

TABLE 5
Box-Behnken test factor level table

| | | | C |
|---|---|---|---|
| | A | B | Adsorption time |
| Level | Buffer pH | Buffer° C. | (T) |
| −1 | 7.5 | 45 | 8 |
| 0 | 8 | 50 | 10 |
| 1 | 8.5 | 55 | 12 |

Response Surface Analysis Test Data of 3 Factors 3 Levels

| | | | | immobilized |
|---|---|---|---|---|
| | | Temperature | | enzyme |
| Serial number | Buffer pH | (° C.) | Time (T) | activity (%) |
| 1 | 7.50 | 50.00 | 7.50 | 1209.54 |
| 2 | 8.50 | 45.00 | 10.00 | 1193.38 |
| 3 | 8.00 | 50.00 | 10.00 | 1605.15 |
| 4 | 7.50 | 55.00 | 10.00 | 1178.67 |
| 5 | 8.00 | 55.00 | 7.50 | 1393.75 |
| 6 | 8.50 | 55.00 | 10.00 | 1426.83 |
| 7 | 8.00 | 50.00 | 10.00 | 1612.83 |
| 8 | 8.00 | 50.00 | 10.00 | 1608.91 |
| 9 | 8.00 | 50.00 | 10.00 | 1610.03 |
| 10 | 8.00 | 45.00 | 12.50 | 1419.49 |
| 11 | 8.00 | 45.00 | 7.50 | 1200.74 |
| 12 | 8.50 | 50.00 | 12.50 | 1368.01 |
| 13 | 7.50 | 45.00 | 10.00 | 1261.4 |
| 14 | 7.50 | 50.00 | 12.50 | 1180.51 |
| 15 | 8.00 | 55.00 | 12.50 | 1312.87 |
| 16 | 8.50 | 50.00 | 7.50 | 1292.65 |
| 17 | 8.00 | 50.00 | 10.00 | 1638.24 |

In summary, adsorption and immobilization of free lipase CAL-A: Firstly, the medium-polarity macroporous resin HPD750 with the highest relative enzyme activity is screened out from 7 kinds of macroporous resins, and then the free CAL-A lipase is immobilized by adsorption method using it as a carrier, and the buffer solution with the highest immobilization relative enzyme activity and immobilization rate is found to be disodium hydrogen phosphate-citric acid. Finally, in order to obtain the optimal adsorption conditions, this application conducted single factor and response surface experiments on the four factors of buffer pH, adsorption temperature, enzyme amount, and adsorption time (Table 7), and combined with factors such as cost, finally determined the optimal adsorption condition. The best adsorption conditions are buffer pH-8.00, adsorption temperature 50.5° C., enzyme dosage 200 mg/g, adsorption time 10.1h, and the activity of immobilized enzyme CAL-A can be 1610.67U/g.

TABLE 7
Analysis of Variance Table of Response Surface Model

| | Sum of | | | | |
|---|---|---|---|---|---|
| Model | squares | Mean | F value | P value | Significance |
| | 4.799E+005 | 53325.52 | 113.36 | <0.0001 | Very |
| | | | | | significant |
| A | 25396.95 | 25396.95 | 53.99 | 0.0002 | |
| B | 7027.64 | 7027.64 | 14.94 | 0.0062 | |
| C | 4241.21 | 4241.21 | 9.02 | 0.0199 | |
| AB | 24992.45 | 24992.45 | 53.13 | 0.0002 | |
| AC | 2724.32 | 2724.32 | 5.79 | 0.0470 | |
| BC | 22444.53 | 22444.53 | 47.71 | 0.0002 | |
| A² | 1.847E+005 | 1.847E+005 | 392.71 | <0.0001 | |
| B² | 83034.63 | 83034.63 | 176.51 | <0.0001 | |
| C² | 85888.04 | 85888.04 | 182.57 | <0.0001 | |
| Residual | 3293.00 | 470.43 | | | |
| value | | | | | |
| Lack of | 2587.84 | 862.61 | 4.89 | 0.0796 | Not |
| fit | | | | | significant |
| Pure error | 705.15 | 176.29 | | | |
| Total | 4.832E+005 | | | | |

### Embodiment 4

After the macroporous resin cross-linked and immobilized lipase, in order to determine the influence of cross-linking conditions on the immobilized enzyme of the present application and find the optimal conditions, the present application carried out single factor and response surface experiments, based on the principle of single variable, related experiments as follows:

Combined with the pre-experimental results, the cross-linking and immobilization operation process is determined according to the response surface data analysis results as follows: After the immobilization of lipase by macroporous resin adsorption, an appropriate concentration (v/v) of cross-linking agent is added immediately, and, shake cross-linking in a shaker at 45° C. and 200 rpm for 4h, the reaction is completed, filter and wash with a buffer with a pH of 8.0, filter to obtain immobilized enzyme, dry overnight, and dry at 40° C. for 4 h, and refrigerate at 4° C. for later use.

1) Screening of Cross-Linking Agents

Take the blank group as the control group, add glutaraldehyde, glyoxal, ethylene glycol diglycidyl ether, and 1,4-butanediol diglycidyl ether at concentrations (v/v) of0.5% and 1.0% respectively to the reaction system after the adsorption test, set up three parallel groups for each group, and carry out the immobilization reaction according to the above-mentioned cross-linking and immobilization conditions, complete the cross-linking reaction after shaking on a constant temperature oscillator at 45° C. and 200 rpm for 4 h, filter and wash with a buffer with a pH of8.0, and obtain the immobilized enzyme by filtration, dry overnight, dry at 40° C. for 4h, and put in a refrigerator at 4° C. for later use.

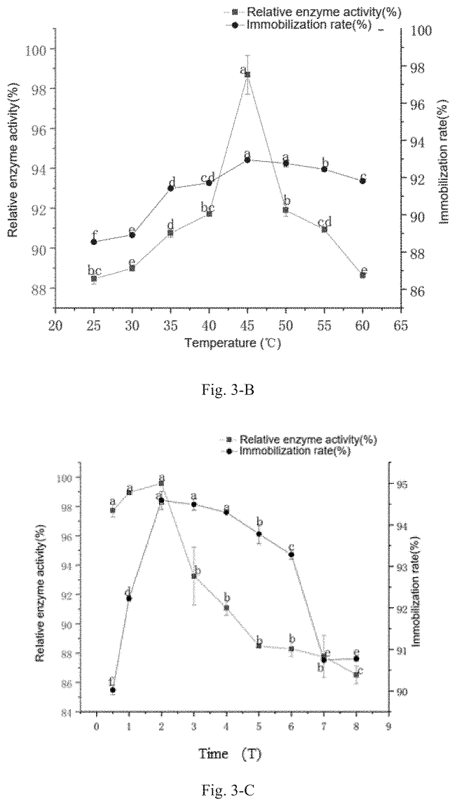

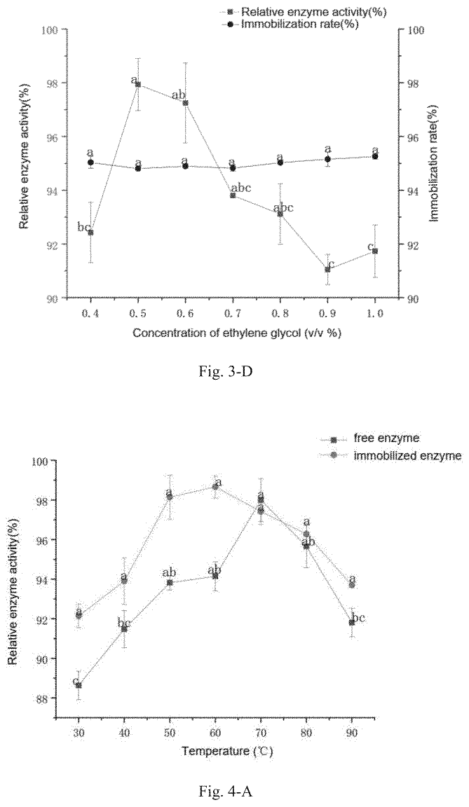

Determine the enzyme activities of different immobilized enzyme (the test method is the same as that in Embodiment 2), data processing, as shown in FIG. 3-A, glyoxal is higher than the relative enzyme activities of immobilized enzymes cross-linked by other cross-linking agents, all above 110%, which is the best choice.

2) Influence of Cross-Linking Temperature on Cross-Linking Immobilization Effect

Immediately add glyoxal with a concentration (v/v %) of0.5% to the reaction system after the adsorption test is completed, set up three parallel groups for each group, and carry out an immobilization reaction according to the above-mentioned cross-linking immobilization conditions, oscillate on a constant temperature oscillator at 25° C., 30° C., 35° C., 40° C., 45° C., 50, 55° C., 60° C., and 200 rpm for 4 hours after cross-linking, complete the reaction, filter and wash with a buffer with a pH of 8.0, filter to obtain the immobilized enzyme, dry overnight, and dry at 40° C. for 4 h, and refrigerate at 4° C. for later use. and placed in a refrigerator at 4° C. for later use. Determine the enzyme activity of different immobilized enzymes, and screen the appropriate cross-linking temperature.

Determine the relative enzyme activity and immobilization rate of different immobilized enzymes (the test method is the same as that in Embodiment 2), data processing, as shown in FIG. 3-B, at 45° C., the relative enzyme activity of the immobilized enzyme is the best, and the immobilization rate are higher than other temperatures, which is the optimal choice.

3) Influence of Cross-Linking Time on Cross-Linking Immobilization

Add glyoxal with a concentration (v/vo) of0.5% to the reaction system after the adsorption test, set up three parallel groups for each group, and carry out the immobilization reaction according to the above-mentioned cross-linking and immobilization conditions, and after cross-linking and oscillating on a constant temperature oscillator at 45° C. and 200 rpm for 0.5h, 1h, 2h, 3h, 4h, 5h, 6h, 7h, and 8h, complete the reaction, filter and wash with a buffer with a pH of 8.0, filter to obtain the immobilized enzyme, dry overnight, and dry at 40° C. for 4 h, and refrigerate at 4° C. for later use. Determine the enzyme activity of different immobilized enzymes, and screen the appropriate cross-linking time.

Measure the relative enzyme activity and immobilization rate of immobilized lipase prepared at different cross-linking times (the test method is the same as that in Embodiment 2), and data processing, as shown in FIG. 3-C, it is the best choice when cross-linking for 2 h.

4) Influence of Cross-Linking Agent Concentration on Cross-Linking Immobilization

Immediately add glyoxal with concentrations (v/v %) of0.4%, 0.5%, 0.6%, 0.7%, 0.8%, 0.9%, and 1.0% to the reaction system after the adsorption test, and set up three parallel groups for each group, carry out the immobilization reaction according to the above cross-linking and immobilization conditions, and complete the cross-linking reaction after shaking on a constant temperature oscillator at 45° C. and 200 rpm for 2 h, filter and wash with a buffer with a pH of 8.0, filter to obtain the immobilized enzyme, and dry overnight, dry at 40° C. for 4 h, and placed in a refrigerator at 4° C. for later use. Determine the enzyme activity of different immobilized enzymes, and screen the appropriate concentration of cross-linking agent.

Measure the relative enzyme activity and immobilization rate of immobilized lipase prepared with different cross-linking agent concentrations (the test method is the same as that in Embodiment 2), data processing, as shown in FIG. 3-D, it is the optimal choice when the cross-linking agent concentration is 0.5% (v/v %).

5) Optimization of Cross-Linking Conditions by Response Surface Experiments

According to the results of the single factor experiment, using the Box-Behnken design principle to conduct a 3 factors 3 levels response surface analysis experiment on the cross-linking agent temperature, cross-linking time, and cross-linking agent concentration to further explore the influence of the three factors on the activity of immobilized lipase. The optimal cross-linking conditions were finally determined, and each group is tested three times in parallel.

TABLE 8
Box-Behnken test factor level table

| | A | B | C |
|---|---|---|---|
| | Cross-linking | Cross-linking | Cross-linking agent |
| Level | temperature (° C.) | temperature (T) | dosage (v/v %) |
| −1 | 40 | 1 | 0.4 |
| 0 | 45 | 2 | 0.5 |
| 1 | 50 | 3 | 0.6 |

TABLE 9
Experimental data of 3 factors and
3 levels response surface analysis

| | Cross-linking | Cross- | Cross-linking | immobilized |
|---|---|---|---|---|
| Serial | temperature | linking | agent | enzyme |
| number | (° C.) | time (T) | dosage (v/v %) | activity (%) |
| 1 | 40.00 | 2.00 | 0.60 | 1658.45 |
| 2 | 45.00 | 2.00 | 0.50 | 1746.69 |
| 3 | 45.00 | 3.00 | 0.60 | 1737.5 |
| 4 | 45.00 | 3.00 | 0.40 | 1663.97 |
| 5 | 50.00 | 3.00 | 0.50 | 1836.76 |
| 6 | 40.00 | 2.00 | 0.40 | 1823.9 |
| 7 | 40.00 | 1.00 | 0.50 | 1656.62 |
| 8 | 45.00 | 2.00 | 0.50 | 1759.56 |
| 9 | 50.00 | 2.00 | 0.40 | 1822.06 |
| 10 | 45.00 | 1.00 | 0.60 | 1818.38 |
| 11 | 50.00 | 2.00 | 0.60 | 1978.31 |
| 12 | 45.00 | 1.00 | 0.40 | 1812.87 |
| 13 | 45.00 | 2.00 | 0.50 | 1713.6 |
| 14 | 45.00 | 2.00 | 0.50 | 1838.6 |
| 15 | 40.00 | 3.00 | 0.50 | 1592.28 |
| 16 | 50.00 | 1.00 | 0.50 | 1879.04 |
| 17 | 45.00 | 2.00 | 0.50 | 1838.6 |

Y=1779.41+98.12A42.05B+8.73C+5.51AB+80.43AC+17.00BC+12.13A²−50.37B²−29.14C²

The extreme point is obtained by deriving the regression equation, the theoretical crosslinking temperature is 49.41, the crosslinking time is 1.76, the amount of crosslinking agent is 0.6%, and the theoretical value is 1984.47 u/g. The activity of the immobilized enzyme obtained in the validation test is 1937.86 u/g.

In summary, the immobilized lipase is immobilized by the CAL-A adsorption-crosslinking method: Select Five different cross-linking agents to compare the enzyme activities of immobilized enzymes, select glyoxal as the best cross-linking agent, and carry out single factor and response surface experiments on the three factors of cross-linking time, cross-linking temperature and cross-linking agent dosage (Table 10). The final optimal cross-linking conditions are as follows: under the conditions of cross-linking temperature of49.4° C., cross-linking time of 1.76 h, and cross-linking agent dosage of 0.6%, the hydrolase activity of immobilized lipase CAL-A is 1937.86 U/g, the immobilization rate is 94.49%.

TABLE 10
Analysis of Variance Table of Response Surface Model

| | Sum of | | | | |
|---|---|---|---|---|---|
| Model | squares | Mean | F value | P value | Significance |
| | 1.331E+005 | 14785.62 | 5.61 | 0.0166 | significant |
| A | 77012.43 | 77012.4 | 29.23 | 0.0010 | significant |
| B | 14145.43 | 14145.43 | 5.37 | 0.0536 | |
| C | 609.70 | 609.70 | 0.23 | 0.6451 | |
| AB | 121.66 | 121.66 | 0.046 | 0.8360 | |
| AC | 25872.72 | 25872.72 | 9.82 | 0.0165 | |
| BC | 1156.68 | 1156.68 | 0.44 | 0.5288 | |
| A² | 619.78 | 619.78 | 0.24 | 0.6425 | |
| B² | 10681.62 | 10681.62 | 4.05 | 0.0839 | |
| C² | 3574.71 | 3574.71 | 1.36 | 0.2822 | |
| Residual | 18442.48 | 2634.64 | | | |
| value | | | | | |
| Lack of | 5639.99 | 1880.00 | 0.59 | 0.6548 | Not |
| fit | | | | | significant |
| Pure error | 12802.49 | 3200.62 | | | |
| Total | 1.515E+005 | | | | |

### Embodiment 5

In order to determine the optimum temperature and optimum pH of immobilized enzyme and free enzyme, and to study the influence of free lipase and immobilized enzyme on the hydrolysis activity of organic solvents, surfactants, metal ions, etc., to find the optimal conditions, the present application has carried out single factor and stability experiments, and the related experiments are as follows:

The optimum temperature and optimum pH of immobilized lipase and free enzyme were studied, and the influences of free lipase and immobilized enzyme on the hydrolysis activity of organic solvent, surfactant, metal ion, etc. were evaluated. Key parameters affecting lipase stability during hydrolyzed triglyceride production were identified, including thermostability, acid-base stability, storage stability, and reusability.

1) The Optimal Temperature of Immobilized Enzyme and Free Enzyme

Place the immobilized CAL-A and free CAL-A in constant temperature water baths at 30, 40, 50, 60, 70, 80, and 90° C. respectively, measure the enzyme activity (the test method is the same as that in Embodiment 2), and the highest enzyme activity is recorded as 100%, determine the optimum temperature.

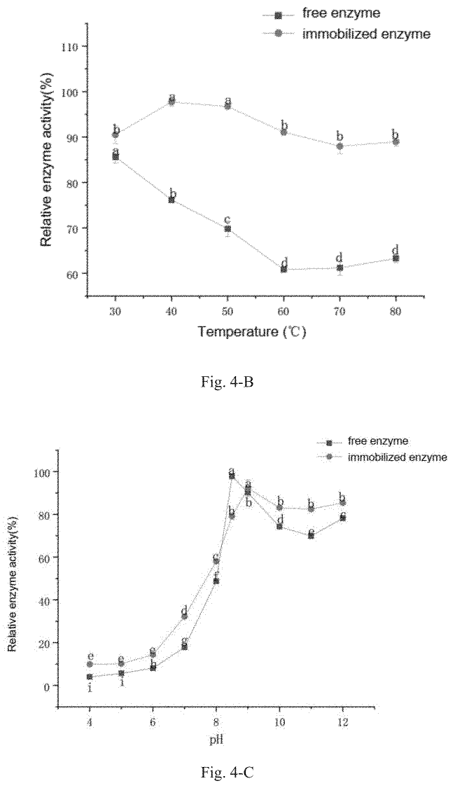

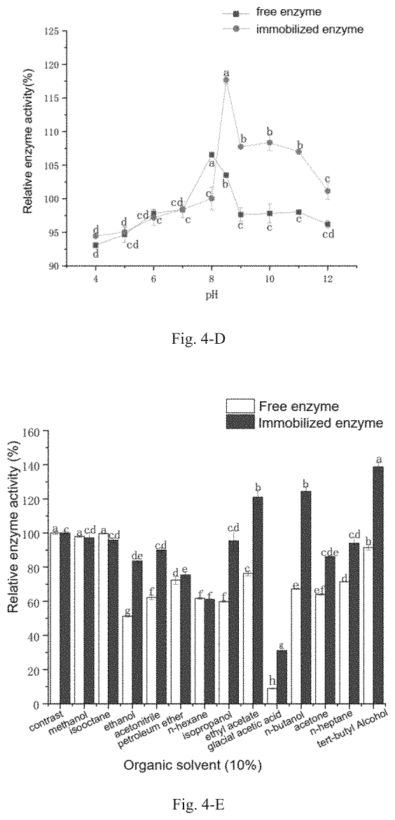

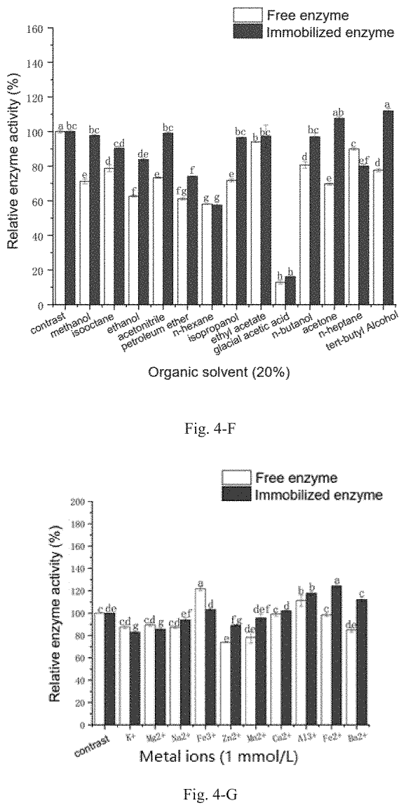

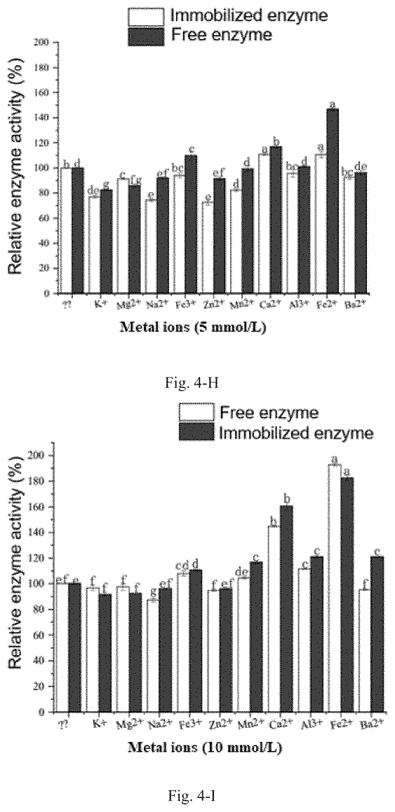

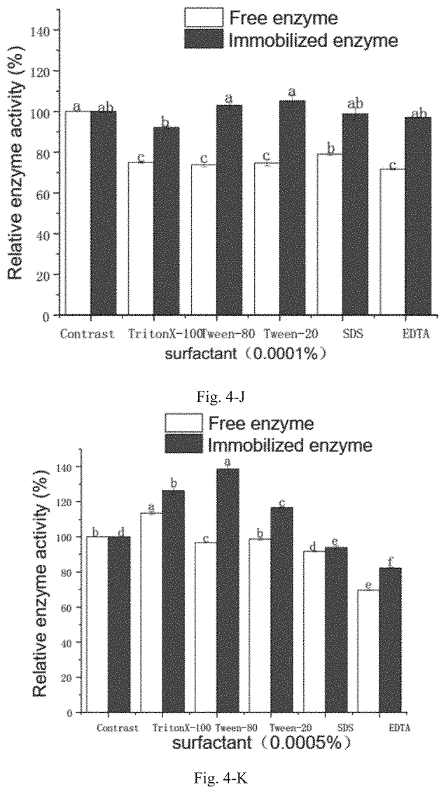

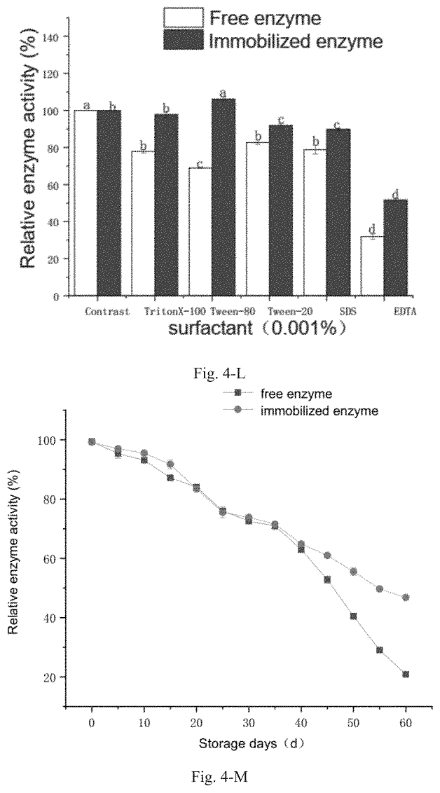

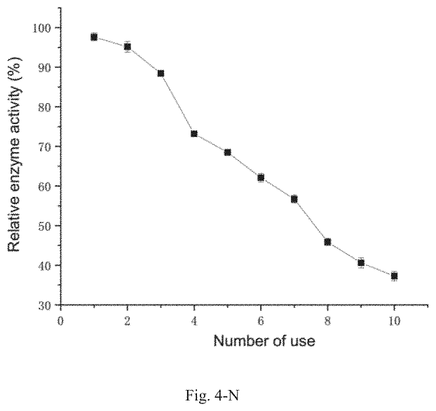

As shown in FIG. 4-A, the optimum temperature of the free enzyme CAL-A is 70° C., and the optimum temperature of the macroporous resin adsorption-crosslinking immobilized enzyme is 60° C.

2) Optimum pH of Immobilized Enzyme and Free Enzyme

Place the immobilized enzyme and the free enzyme in the substrate prepared by the buffer with a pH of 4, 5, 6, 7, 8, 9, 10, 11, and 12 respectively, measure the enzyme activity, and the highest enzyme activity is recorded as 100%, determine the optimum pH.

As shown in FIG. 4-C, the optimum pH value of the free enzyme is 8.5, while the optimum pH value of the immobilized enzyme is 9.

3) Influence of Metal Ions on Immobilization

Add 10 mmol/L, 5 mmol/L, l0 mmol/L K+, Al³⁺, Fe²⁺, Fe3+, Ca²⁺, Mg²⁺, Mn²⁺, Zn²⁺, Ba²⁺, Na²⁺ and other 10 kinds of metal ion solutions to measure the enzyme activity respectively, take the activity of the untreated immobilized enzyme CAL-A and free enzyme CAL-A enzyme as 100%, analyze the influence of metal ions on the enzyme activity. (FIG. 4-G, FIG. 4-H, FIG. 4-I).

4) Influence of Organic Solvent on Immobilized Enzyme

Add 10% (v/v) and 20% (v/v) methanol, ethanol, acetonitrile, acetone, n-butanol, isooctane, isopropanol, n-hexane, n-heptane, petroleum ether, tert-butyl Alcohol, glacial acetic acid, ethyl acetate and other 13 kinds of organic solvent solutions respectively, measure the enzyme activity. Take the untreated immobilized enzyme CAL-A and free enzyme CAL-A enzyme activity as 100%, analyze the influence of organic solvent on the enzyme activity.

As shown in FIG. 4-E and FIG. 4-F, ethanol, petroleum ether, n-hexane, and acetic acid can significantly inhibit the hydrolytic activity, while ethyl acetate, n-butanol, and tert-butanol can activate the immobilized enzyme.

5) Influence of Surfactant on Immobilization

Add 0.0001%, 0.0005%, 0.001% SDS, TritionX-100, Tween-80, Tween-20, EDTA and other 5 kinds of surfactant solutions respectively, measure the enzyme activity, take the untreated immobilized enzyme CAL-A and free enzyme CAL-A enzyme activity as 100%, analyze the influence of surfactant on enzyme activity.

As shown in FIG. 4-J, FIG. 4-K, and FIG. 4-L, 0.0005% EDTA in the surfactant had an activating effect on lipase, while 0.001% EDTA had an inhibitory effect.

6) Thermal Stability of Immobilized Enzyme and Free Enzyme

Place the immobilized enzyme and free enzyme in constant temperature water baths at 30, 40, 50, 60, 70, 80, and 90° C. for 2 h, measure the enzyme activity after taking it out, record the highest enzyme activity as 100%, and analyze its thermal stability (FIG. 4-B).

7) pH Stability of Immobilized Enzyme and Free Enzyme

Place the immobilized enzyme and free enzyme in the substrate prepared by the buffer with pH of4, 5, 6, 7, 8, 9, 10, 11,12 for 2 h, measure the enzyme activity after taking it out, record the highest enzyme activity as 100%, analyze the pH stability (FIG. 4-D).

8) Reuse Stability

Catalyze the immobilized enzyme and the substrate under the optimal reaction conditions to measure the enzyme activity. After the reaction, separate the immobilized enzyme from the reaction system, wash the liquid in the test tube, wash three times with buffer, suck up the washing solution, and repeat the above reaction ten times in total. Take the enzyme activity measured for the first time as 100%, study the reusability, as shown in FIG. 4-N.

9) Storage Stability

Seal he prepared immobilized enzyme and store in a refrigerator at 4° C. for 60 days, and measure the activity of the immobilized enzyme (4-M) every 5 days.

In summary, the results of research on the properties of the fre enzyme and the immobilized enzyme CAL-A: In terms of temperature: the optimum temperature of the free enzyme CAL-A is 70° C., and the optimum temperature of the macroporous resin adsorption-crosslinking immobilized enzyme is 60° C., although the optimum temperature is lower than that of the free enzyme CAL-A, but the thermal stability has been improved. The macroporous resin adsorption-crosslinking immobilized enzyme can still maintain 90% of the activity of immobilized CL-A at 80° C., while the free enzyme only has 64.71%. n terms of pH value: in an environment with a pH value of 4 to 12, it was found that the most suitable pH value of the free enzyme was 8, while the most suitable pH value of the immobilized enzyme was 8.5, and the acid-base stability was also improved. In terms of organic solvents: ethanol, petroleum ether, n-hexane, and acetic acid have obvious inhibitory effects on hydrolysis activity, while ethyl acetate, n-butanol, and tert-butanol have activation effects on immobilized enzymes. In terms of metal ions and surfactants: metal ions Fe²⁺ can activate the hydrolysis of immobilized enzymes. During the experiment, contact with Zn²⁺, Mn²⁺, and Ba²⁺ should be avoided; 0.0005% EDTA in the surfactant can activate lipase, while 0.001% EDTA can inhibit it, and the immobilized enzyme has better operation stability properties and storage stability.

### Embodiment 6

In this Embodiment, the influences of the optimum hydrolysis time, the optimum hydrolysis temperature, the amount of water and the amount of enzyme on the reaction of the diglyceride prepared in Embodiment 1 were investigated. Secondly, the optimal experimental process was obtained by response surface design, and the content of diglyceride was detected by HPLC-ELSD method.

Specific steps are as follows:

1. Analysis of Composition of Fatty Acid Using Gas Phase

1.1 Methyl Esterification Method

Refer to the national standard GB5009.168-2016 “Determine fatty acids in food” to modify the steps of methyl esterification, and use gas chromatography to detect the composition of fatty acids in oils.

In the detection of fatty acids in oils, fatty acid methyl esters are prepared by esterification of fatty acids, and then analyzed and detected by gas chromatography. First, add 0.01 g of oil sample to 2 mL of 2% NaOH-methanol solution, put it into a 10 mL glass bottle with a cover, and put it in a water bath at 80° C. for 10 min, then add 1.75 mL of boron trifluoride-methanol solution, and heat it at 80° C. in a water bath for 2 minutes. Rinse the above solution with running water and cool to room temperature, then accurately add 1 mL of n-hexane, shake for 2 min, then add 2 mL of saturated NaCl-water solution, centrifuge at 3000r/min to take the supernatant for filtration and detection.

1.2 Detection Conditions of Gas Chromatography

DB-23 capillary column with column length of 30 m, an inner diameter of 0.25 mm and a film thickness of0.25 um, and a hydrogen flame ionization detector. The inlet and detector temperatures are 250° C. and 280° C., respectively. Heating program: keep at 50° C. for 1 min, then rise to 175° C. at 25° C./min and hold for 1 min, finally rise to 230° C. at 4° C./min and hold for 5 min, the carrier gas is hydrogen with a purity greater than 99.99%: the injection volume is 1 UL, the split ratio is 50:1. Set the nitrogen flow rate to 30 mL/min, the air flow rate to 450 mL/min, and the hydrogen flow rate to 40 mL/min.

1.3 Qualitative Analysis of Fatty Acids

Measure 37 kinds of fatty acid standard substances, compare and analyze the chromatograms of the standard substance and the analyte, and determine the type of fatty acid represented by each chromatographic peak in the chromatogram of the tested sample.

2. HPLC-ELSD Analysis of Glyceride Composition

2.1 Liquid Chromatography Conditions

Chromatographic column: ChromSpher 5 Lipids column, 250×4.6 mm (part number 28313); column temperature: 30° C.; mobile phase (A): n-hexane-isopropanol-ethyl acetate (820:40:140), mobile phase (B): n-hexane-isopropanol-acetonitrile (956:40:4); injection volume: 10 μL; flow: 0.8 mL/min; before the next injection, the HPLC system was kept in the initial mobile phase for 2 min, to ensure the stability of the injection. The elution program is shown in the table below.

TABLE 11
Liquid chromatography mobile phase and
gradient elution gradient conditions

| Time(min) | Mobile phase A (%) | Mobile phase B (%) |
|---|---|---|
| 0.0 | 100 | 0 |
| 8 | 100 | 0 |
| 15 | 0 | 100 |
| 18 | 100 | 0 |
| 40 | 100 | 0 |

Atomizer pressure: 35 psi; Atomizer flow rate: 6 L/min; Atomizer temperature: 36° C.; ELSD drift tube temperature: 40° C.; Pressure: 2.5*105 Pa.

2.2 Sample Pretreatment Process

Weigh 100 ul sample and place it in a 10 mL centrifuge tube, add 4 mL ether, elute to remove free fatty acid, vortex for 1 min to mix well, carry out chromatography on alumina, then remove ether with nitrogen blower, and finally add 900 uL n-hexane to constant volume, sonicate for 1 min, vortex for 1 min, filter with 0.45 u filter membrane, and detect by HPLC. All samples were measured three times, and the final results were represented by the average value of the three times.

2.3 Preparation of 1,3 Diacylglycerol by Hydrolysis

Mix the high oleic sunflower oil and immobilized lipase CAL-A with deionized water in a 25 mL triangular stoppered bottle uniformly, dissolve at a suitable temperature, and put into a constant temperature water bath shaker for reaction. Determine the optimal reaction time, reaction temperature, enzyme amount and water amount.

2.3.1 Influence of Reaction Time on the Hydrolysis of 1,3 Diacylglycerol

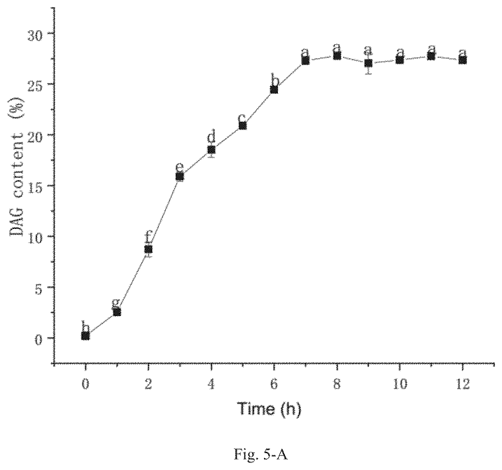

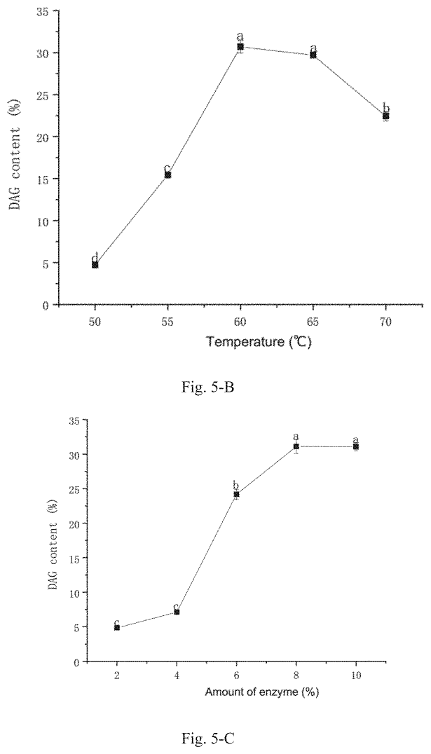

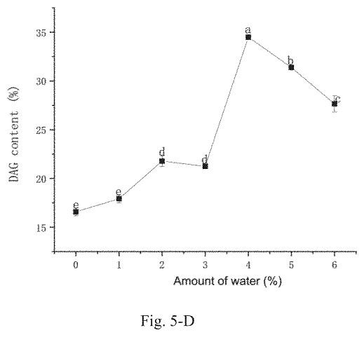

Accurately weigh 5 g of high oleic sunflower oil, mix the immobilized lipase CAL-A (accounting for 6% of the oil weight) and deionized water (4%) evenly in a 25 mL triangular stoppered bottle, and mix and hydrolyze at 60° C., and then put it into a constant temperature water bath shaker for hydrolysis reaction. Take samples at 0, 1, 2, 3, 4, 5, 6, 7, 8, 9, 10, 11, and 12 h, and detect the diglyceride content by HPLC-ELSD method, as shown in FIG. 5-A, at 7 hours, the DAG hydrolysis reaction is basically complete.

2.3.2 Influence of Reaction Temperature on Hydrolysis of 1,3 Diacylglycerol

Accurately weigh 5 g of high oleic sunflower oil, mix the immobilized lipase CAL-A (accounting for 6% of the oil weight) and deionized water (4%) evenly in a 25 mL triangular stoppered bottle, and mix and hydrolyze at 50° C., 55° C., 60° C., 65° C., and 70° C., take a sample after 7 hours of reaction, and then put it in a constant temperature water bath shaker for hydrolysis reaction, and detect the diglyceride content by HPLC-ELSD method, as shown in the FIG. 5-B, at 60° C., the DAG content reaches more than 30%, and the optimum hydrolysis reaction temperature is 60° C.

2.3.3 Influence of the Amount of Enzyme on the Hydrolysis of 1,3 Diacylglycerol

Accurately weigh 5 g of high oleic sunflower oil, mix the immobilized lipase CAL-A which accounts for 2%, 4%, 6%, 8%, and 10% of the oil weight with deionized water (4%) evenly in a 25 mL triangular stoppered bottle, mix and hydrolyze at 60° C., take a sample after 7 hours of reaction, put it in a constant temperature water bath shaker for hydrolysis reaction, and detect the diglyceride content by HPLC-ELSD method, as shown in FIG. 5-C, when the amount of enzyme accounts for 8% of the oil weight, the hydrolysis effect is the best.

Influence of the Amount of Water on Hydrolysis of 1,3 Diacylglycerol

Accurately weigh 5 g of high oleic sunflower oil, mix immobilized lipase CAL-A which accounts for 8% of the oil weight with deionized water (0%, 1%, 2%, 3%, 4%, 5%, 6%) evenly in a 25 mL triangular stopper bottle, mix and hydrolyze at 60° C., take a sample after 7 hours of reaction, place in a constant temperature water bath shaker for hydrolysis reaction, and detect the diglyceride content by HPLC-ELSD method, as shown in FIG. 5-D, when the amount of water accounts for 4% of the oil weight, the hydrolysis effect is the best.

2.4 Hydrolysis Conditions Optimized by Response Surface Methodology

According to the results of the single factor experiment, using the Box-Behnken design principle, a response surface analysis experiment with 4 factors and 3 levels was carried out on the reaction time, reaction temperature, the amount of enzyme and the amount of water. The Box-Behnken test factor level table is shown in the table below.

TABLE 12
Box-Behnken test factor level table

| | A | B | C | D |
|---|---|---|---|---|
| | Reaction | Reaction | Amount of | Amount |
| | temperature | time | enzyme | of water |
| Level | ° C. | (T) | (v/v %) | (v/v %) |
| −1 | 57.5 | 6.5 | 7 | 3.5 |
| 0 | 60 | 7 | 8 | 4 |
| 1 | 62.5 | 7.5 | 9 | 4.5 |

The content of diglyceride in oil was analyzed HPLC-ELSD method. At the same time, the four factors of hydrolysis time, hydrolysis temperature, enzyme amount and water addition amount were tested by single factor and response surface (Table 13), and the regression equation was obtained. The extreme point was obtained by deriving the regression equation, and the optimal theoretical hydrolysis temperature was 62.44° C., the hydrolysis time was 7.5, the amount of enzyme was 8.91, and the amount of water was 3.72. Under this condition, the DAG content can reach 36.12%.

TABLE 13
Analysis of Variance Table of Response Surface Model

| | Sum of | | | | |
|---|---|---|---|---|---|
| Model | squares | Mean | F value | P value | Significance |
| | 275.30 | 19.66 | 7.29 | 0.0007 | significant |
| A | 4.40 | 4.40 | 1.63 | 0.2256 | |
| B | 108.54 | 108.54 | 40.21 | <0.0001 | |
| C | 94.87 | 94.87 | 35.15 | <0.0001 | |
| D | 12.77 | 12.77 | 4.73 | 0.0503 | |
| AB | 0.86 | 121.66 | 0.046 | 0.8360 | |
| AC | 1.49 | 1.49 | 0.55 | 0.4720 | |
| BC | 3.80 | 3.80 | 1.41 | 0.2582 | |
| BD | 29.65 | 29.65 | 10.98 | 0.0062 | |
| CD | 0.44 | 0.44 | 0.16 | 0.6949 | |
| A² | 3.34 | 3.34 | 1.24 | 0.2878 | |
| B² | 4.68 | 4.68 | 1.73 | 0.2127 | |
| C² | 1.89 | 1.89 | 0.70 | 0.4193 | |
| D² | 1.25 | 1.25 | 0.46 | 0.5083 | |
| Residual | 32.39 | 2.70 | | | |
| value | | | | | |
| Lack of | 27.04 | 2.70 | 1.01 | 0.5944 | Not |
| fit | | | | | significant |
| Pure error | 5.35 | 2.67 | | | |
| Total | 307.69 | | | | |

What is claimed is:

1. A method for preparing diacylglycerols by immobilized Sn-2 lipase, comprising the steps:
S1 Adsorb Sn-2 lipase with resin: pipette original enzyme solution of Sn-2 lipase in a centrifuge tube, add pretreated wet carrier macroporous resin, shake and absorb in a water bath, and a reaction is completed after dispersion and uniformity, air-dry overnight after suction filtration, and dry to obtain Sn-2 lipase adsorbed by macroporous resin;
S2 Cross-linking and immobilization of Sn-2 lipase: after resin adsorption is completed, add a cross-linking agent, oscillate for cross-linking filter, wash with buffer, filter, dry, and refrigerate to obtain immobilized Sn-2 lipase macroporous resin;
S3 Hydrolysis: Add immobilized Sn-2 lipase macroporous resin to the oil raw material, and obtain 1,3-diglycerol by one-step hydrolysis.

2. The method for preparing diacylglycerols by immobilized Sn-2 lipase according to claim 1, wherein in S1, a pretreatment method of the macroporous resin is: soak in 95% absolute ethanol for 24h, stir continuously to make it dissolve completely, vacuum filtration, rinse with distilled water until the water is no longer turbid and there is no obvious ethanol smell, wash away the residual impurities and absolute ethanol, soak in deionized water and seal at 4° C. for storage for later use.

3. The method for preparing diacylglycerols by immobilized Sn-2 lipase according to claim 1, wherein in S1, the model of macroporous resin is any one of RDK09, DA201, LXT-008, HPD700, HPD100, X-5, HPD750.

4. The method for preparing diacylglycerols by immobilized Sn-2 lipase according to claim 1, wherein in S1, conditions for water-bath shaking adsorption are: shake at 250r/min on a water-bath shaker for 12h at 25-70° C.

5. The method for preparing diacylglycerols by immobilized Sn-2 lipase according to claim 1, wherein in S1, conditions of the adsorption are: pH of the buffer solution is 5.5˜9, an adsorption temperature is 35˜60° C., the amount of enzyme is 100˜400 mg/g, and an adsorption time is 10˜14h.

6. The method for preparing diacylglycerols by immobilized Sn-2 lipase according to claim 1, wherein in S2, the cross-linking agent is any one of glutaraldehyde, glyoxal, oxalic acid diglycidyl ether, 1,4 butyl glycol diglycidyl ethers, wherein the volume concentration of the cross-linking agent is 0.5%.

7. The method for preparing diacylglycerols by immobilized Sn-2 lipase according to claim 1, wherein in S2, conditions of the cross-linking are: cross-linking temperature 35˜55° C., cross-linking time 2˜6h, cross-linking agent dosage 0.5˜0.7% (volume).

8. The method for preparing diacylglycerols by immobilized Sn-2 lipase according to claim 1, wherein in S2, the buffer is any one of disodium hydrogen phosphate-citric acid buffer, potassium dihydrogen phosphate-sodium hydroxide buffer, disodium hydrogen phosphate-sodium dihydrogen phosphate buffer, barbiturate-hydrochloric acid buffer, tris hydrochloride buffer, glycine-sodium hydroxide buffer.

9. The method for preparing diacylglycerols by immobilized Sn-2 lipase according to claim 1, wherein in S3, the oil raw material is vegetable oil.

10. The method for preparing diacylglycerols by immobilized Sn-2 lipase according to claim 1, wherein in S3, conditions of the hydrolysis are: hydrolysis temperature 60˜65° C., hydrolysis time 7˜8h, the amount of enzyme 6˜10 % (volume), the amount of water added 3.5˜6% (volume).
